(19) United States
(12) Patent Application Publication
(10) Pub. No.: US 2021/0378336 A1
(43) Pub. Date: Dec. 9, 2021

(54) PERSPIRATION SHIELD

(71) Applicant: TNH Enterprises Limited, Milton Keynes (GB)

(72) Inventors: Theresa Jane Pope, Milton Keynes (GB); Hayley Marie Leete, Northampton (GB); Neil Leete, Northampton (GB)

(21) Appl. No.: 16/649,658

(22) PCT Filed: Sep. 20, 2018
(86) PCT No.: PCT/GB2018/000124

§ 371 Date: Mar. 22, 2020

(30) Foreign Application Priority Data
Sep. 21, 2017 (GB) 1715285.1

(51) Int. Cl. A41D 27/13

(52) U.S. Cl. CPC A41D 27/133

(57) ABSTRACT

A perspiration shield (101) comprises reinforcing layer (203), perspiration impermeable layer (204), perspiration absorbent layer (205) and liner layer (206). The perspiration impermeable layer (204) is provided with an adhesive composition (215) disposed on a border (214) of its inner face (213), forming an adhesive region (216). The perspiration absorbent layer (205) comprises a perspiration absorbent material which is arranged on the inner face (211) of the perspiration impermeable layer (204). The reinforcing layer (203) and the liner layer (206) are releasably attached to the perspiration impermeable layer (204) and the perspiration absorbent layer (205) respectively. The liner layer (206) and the reinforcing layer (203) are releasable prior to and following respectively adhering of the shield (101) to the skin of a wearer's (102) underarm about the adhesive region (216). The shield (101) obviates issues in relation to skin irritation and poor adhesiveness whilst providing a shield (101) that is more easily applied and is sufficiently stable to be stored prior to use.

### BACKGROUND OF THE INVENTION

### 1. Field of the Invention

The present invention relates to perspiration shields that are releasably adherable to the skin of an underarm.

### 2. Description of the Related Art

Perspiration shields for releasably adhering to the skin of a wearer's underarm are known. Typically such shields include a perspiration absorbent material, for example cotton fibres, forming an inner, skin facing layer, to absorb perspiration excreted by the underarm sweat gland area. Additionally, it is common for such shields to include a perspiration impermeable outer layer, in order to prevent perspiration absorbed by the absorbent material from leaking onto the wearer's clothing, as might otherwise occur by capillary action of the absorbent material or in the event that the absorbent material were to become saturated. The shields are often adhered to the skin of the wearer's underarm by way of a pressure sensitive, perspiration resistant, adhesive composition applied over an area of the skin-facing surface of the shield.

It is desirable, both to improve comfort for the wearer and to reduce leakage of perspiration from the underarm past the shield, that the shield conforms closely to the shape of the wearer's underarm. More particularly, it is desirable that the inner perspiration absorbing surface of the shield is maintained in contact with the skin of the wearer's underarm. To achieve the desired close conformation of the absorbent surface of the shield, it is required that the shield is correctly applied to the underarm skin by the wearer in the first instance, such that the adhesive composition on the skin-facing surface adheres to the correct region of the underarm skin. Further, to achieve the desired close-conformance and wearer comfort, it is required that the shield is relatively flexible to allow it to flex during use with movement of the wearer. However, it is desirable that undue flexing of the shield during its application to the skin is minimised to facilitate correct positioning of the shield with respect to the wearer's underarm, and to prevent creasing or wrinkling of the shield during application.

Further, it is desirable that that shield comprises an adhesive composition that does not irritate the wearer's underarm skin, even during periods of prolonged use.

Moreover, it is desirable that the shield is chemically and mechanically stable before use, such that the shield once manufactured may be stored awaiting use without degrading or otherwise losing its functional efficiency.

It is desirable therefore to provide a perspiration shield which obviates the above noted problems that can be encountered with perspiration shields of the prior art. In particular, it is desirable to provide a shield with means for facilitating correct application of the shield to the skin of a wearer's underarm, and/or to provide a shield with an adhesive that does not irritate the skin of a wearer's underarm, and/or to provide a shield which is mechanically and chemically stable to facilitate storage of the shield prior to use.

### BRIEF SUMMARY OF THE INVENTION

According to a first aspect of the present invention, there is provided a perspiration shield releasably adherable to the skin of an underarm, the shield having an inner face and an outer face, the shield comprising: a perspiration impermeable layer having an inner face, an outer face, and a peripheral edge, and comprising an adhesive composition suitable for releasably adhering said shield to the skin of an underarm disposed on at least a region of said inner face thereof forming an adhesive region; a perspiration absorbent material arranged on said inner face of said perspiration impermeable layer; and a reinforcing layer having an inner face, an outer face, and a peripheral edge, said reinforcing layer being arranged on said outer face of said perspiration impermeable layer and extending at least partly thereacross, with at least a portion of said inner face of said reinforcing layer in contact with and releasably attached to said outer face of said perspiration impermeable layer; in which said reinforcing layer defines radially protruding regions and radially concaved regions bounded by said peripheral edge thereof, said radially protruding regions extending radially outwardly of the centre of said reinforcing layer to a greater extent than said radially concaved regions.

Preferably, said radially protruding regions extend radially outwardly of the centre of the reinforcing layer to an extent that the peripheral edge of said reinforcing layer at the greatest extent of said radially protruding regions is substantially aligned with said peripheral edge of said perspiration impermeable layer.

Preferably, said radially concaved regions extend radially outwardly of the centre of the reinforcing layer to an extent that the peripheral edge of said reinforcing layer at the minimum extent of said radially concaved regions is aligned with a part of said perspiration impermeable layer radially inward of said peripheral edge of said perspiration impermeable layer.

Preferably, substantially the full area of said inner face of said reinforcing layer is in contact with said outer face of said perspiration impermeable layer.

Preferably, said inner face of said reinforcing layer is in contact with and releasably attached to said outer face of said perspiration impermeable layer adjacent the peripheral edge of said reinforcing layer.

Preferably, said inner face of said reinforcing layer is in contact with and releasably attached to said outer face of said perspiration impermeable layer adjacent the peripheral edge of said reinforcing layer at the maximum extent of said radially protruding regions adjacent the peripheral edge of said perspiration impermeable layer.

Preferably, said inner face of said reinforcing layer is in contact with and releasably attached to said outer face of said perspiration impermeable layer adjacent substantially the full length of the peripheral edge of said reinforcing layer.

Preferably, substantially the full area of said inner face of said reinforcing layer is in contact with and releasably attached to said outer face of said perspiration impermeable layer.

Preferably, said reinforcing layer is relatively stiffer than said perspiration impermeable layer.

Preferably, said perspiration impermeable layer is approximately a polygon in shape, the peripheral edge thereof defining generally faces and vertices forming a closed loop.

Preferably, said reinforcing layer and said perspiration impermeable layer are relatively oriented such that at least one of said radially concaved regions of said reinforcing layer is aligned with at least one of said vertices of said perspiration impermeable layer.

Preferably, said reinforcing layer and said perspiration impermeable layer are relatively oriented such that a pair of said radially concaved regions are height wise aligned along opposed height wise extending sides of said perspiration impermeable layer.

Preferably, said reinforcing layer and said perspiration impermeable layer are relatively oriented such that a pair of said radially concaved regions are height wise aligned along an axis extending width wise of the shield approximately half the height of the shield along opposed height wise extending sides of said perspiration impermeable layer.

Preferably, said reinforcing layer comprises first and second discrete portions.

Preferably, said shield is approximately triangular in shape having a width wise extending base side, and a pair of generally height wise extending sides.

Preferably, said perspiration absorbent material is permanently attached to said inner face of said perspiration impermeable material in a way that said perspiration absorbent material is substantially prevented from detaching therefrom during normal use of the shield.

Preferably, said reinforcing layer is releasably attached to said perspiration impermeable layer by attachment means which function to allow said reinforcing layer to be readily detached from said perspiration impermeable layer substantially without damage to said perspiration impermeable layer and leaving substantially no adhesive residue on said outer face of said perspiration impermeable layer.

Preferably, said perspiration absorbent material defines a discrete layer of said shield.

Preferably, each of said perspiration impermeable layer, said perspiration absorbent layer, and said barrier layer are generally planar flexible sheets.

Preferably, said shield is substantially acute-isosceles triangular in shape.

Preferably, said perspiration impermeable layer comprises a polyurethane film.

Preferably, said perspiration absorbent material comprises viscose fibre.

Preferably, said perspiration shield further comprises a liner layer having an inner face, an outer face, and a peripheral edge, said liner layer being arranged such that said outer face thereof is in contact with said inner face of said perspiration impermeable layer and extends substantially fully across said adhesive region thereof so as to substantially fully cover said adhesive region thereof.

According to a second aspect of the present invention there is provided a perspiration shield releasably adherable to the skin of an underarm, the shield having an inner face and an outer face, the shield comprising: a perspiration impermeable layer having an inner face, an outer face, and a peripheral edge, and comprising an adhesive composition suitable for releasably adhering said shield to the skin of an underarm disposed on at least a region of said inner face thereof forming an adhesive region; a perspiration absorbent material arranged on said inner face of said perspiration impermeable layer; and a liner layer having an inner face, an outer face, and a peripheral edge, said liner layer being arranged such that said outer face thereof is in contact with said inner face of said perspiration impermeable layer and extends substantially fully across said adhesive region thereof so as to substantially cover said adhesive region thereof; in which said liner layer comprises an ethylene based plastics material.

Preferably, said liner layer consists of a plastics material comprising a major proportion of the ethylene monomer.

Preferably, said inner face of said ethylene based plastics liner layer is substantially free of coating or contamination and said inner face is defined by said ethylene plastics material.

Preferably, said liner layer comprises a low-density polyethylene (LDPE) material.

Preferably, said liner layer comprises a generally planar sheet of low-density polyethylene material defining said inner face thereof, and in which said inner face is substantially free of coating or contamination.

Preferably, said liner layer is flexible.

Preferably, said adhesive composition comprises a silicone based adhesive substance.

According to a third aspect of the present invention there is provided a perspiration shield releasably adherable to the skin of an underarm, the shield having an inner face and an outer face, the shield comprising: a perspiration impermeable layer having an inner face, an outer face, and a peripheral edge, and comprising an adhesive composition suitable for releasably adhering said shield to the skin of an underarm disposed on at least a region of said inner face thereof forming an adhesive region; a perspiration absorbent material arranged on said inner face of said perspiration impermeable layer; in which said adhesive composition comprises a silicone based adhesive substance.

Preferably, said adhesive composition is disposed on said inner face of said perspiration impermeable layer adjacent the peripheral edge thereof defining a border circumferential about said perspiration absorbing material.

Preferably, said perspiration shield further comprises a liner layer having an inner face, an outer face, and a peripheral edge, said liner layer being arranged such that said outer face thereof is in contact with said inner face of said perspiration impermeable layer and extends substantially fully across said adhesive region thereof so as to substantially cover said adhesive region thereof; in which said liner layer comprises an ethylene based plastics material.

Preferably, said liner layer consists of a plastics material comprising a major proportion of the ethylene monomer.

Preferably, said inner face of said ethylene based plastics liner layer is substantially free of coating or contamination and said inner face is defined by said ethylene plastics material.

Preferably, said liner layer comprises a low-density polyethylene (LDPE) material.

Preferably, said liner layer comprises a generally planar sheet of low-density polyethylene material defining said inner face thereof, and in which said inner face is substantially free of coating or contamination.

Preferably, said liner layer and said perspiration impermeable layer are adapted so as to be readily separable.

### BRIEF DESCRIPTION OF THE DRAWINGS

The invention will now be described by way of example only with reference to the accompanying drawings, which are purely schematic and not to scale, of which:

FIG. 1 shows an exemplary embodiment of a perspiration shield according to the present invention;

FIGS. 2a, 2b, 2c, 2d and 2e show the perspiration shield 101 in inner and outer plan, side and end elevations, and an exploded view respectively;

FIGS. 2a, 2b, 2c, 2d, 2e, 2f, 2g and 2h show the perspiration shield in outer and inner plan views, in face and end elevation views, in face and end elevation exploded views, and in partial inner and outer plan views respectively. FIG. 2i shows the reinforcing layer of the perspiration shield in isolation and FIG. 2j shows the layers of the perspiration shield.

FIGS. 3a and 3b show the perspiration shield in first and second side elevation views respectively. The perspiration shield is shown in FIGS. 3c and 3d attached to the skin of a wearer's underarm with the reinforcing layer attached and partially detached respectively.

FIG. 4 shows the perspiration shield being detached from the wearer's underarm skin.

### DETAILED DESCRIPTION OF EXAMPLE EMBODIMENTS

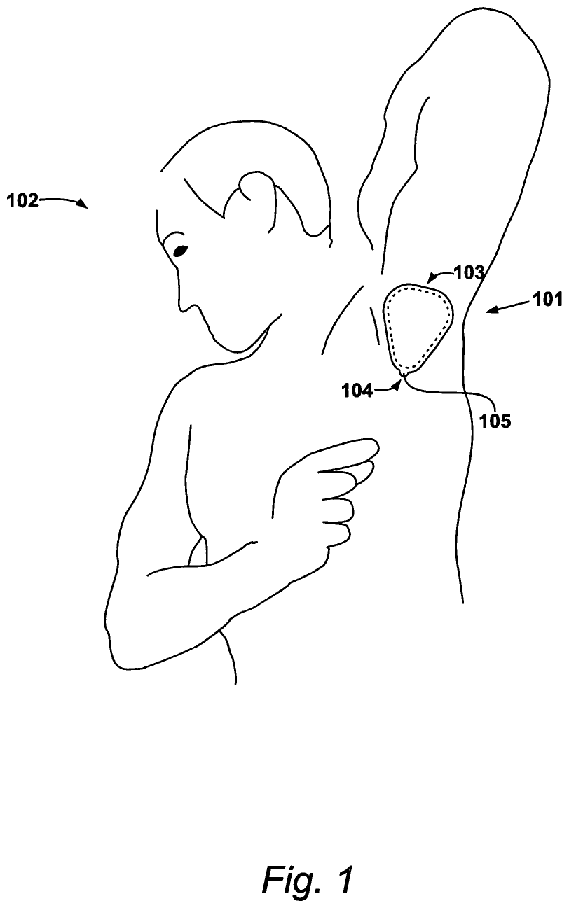

### FIG. 1

An exemplary embodiment of a perspiration shield according to the present invention is illustrated in FIG. 1.

In the Figure, a perspiration shield 101 according to an embodiment of the present invention is shown adhered to the skin of the underarm of the wearer 102. As will be appreciated, the primary function of said perspiration shield 101 is to absorb and contain perspiration excreted by the underarm sweat gland area, so as to prevent the perspiration from coming into contact with the wearer's clothing, and moreover to prevent the release of unpleasant odours produced by bacteria metabolizing chemicals present in the perspiration. The perspiration shield 101 is configured to be releasably adherable to the wearer's underarm skin, in such a way as to allow the wearer to readily adhere or remove the shield from the skin. In the specific embodiment, the shield 101 is configured to be single-use, and thus it is expected that the shield will be adhered to and removed from the skin only once before being discarded.

As illustrated, in the embodiment the shield 101 is generally triangular in shape. More particularly, in the specific embodiment illustrated the shield 101 is generally acute-isosceles triangular in shape. As used herein, the term ‘acute-isosceles triangular’ is intended to define the shield as being generally triangular in shape and having only two faces of generally equal length, and wherein the vertex angle, and indeed all three angles, are acute.

In the embodiment, the shield defines an arm end 103 and a torso end 104. The arm end 103 is relatively wider than the torso end 104. In the particular embodiment, the torso end 104 is defined generally by the vertex of the generally triangular shield, and the arm end 103 is defined generally by the base of the generally triangular shaped shield.

As illustrated, in the specific embodiment said shield 101 is configured for wearing by the wearer in an inverted orientation, i.e. in an orientation wherein the base of the triangular shield is oriented generally towards the arm of the wearer, and the vertex of the shield is oriented generally towards the torso of the wearer.

The particular acute-isosceles triangular shape of the present shield 101 is advantageous as it approximates closely to the average shape of a person's underarm sweat gland area. Moreover, the present applicant is aware that the underarm sweat gland area is usually generally acute-isosceles triangular in shape, with the area being relatively great in width towards the arm, and then tapering in width relatively sharply towards the torso almost to a point. Thus, the inverted orientation of the present shield 101, along with the shape of the shield 101, permits near full coverage of the sweat gland area with a near minimum area of shield material. In this respect, it will be appreciated that it is desirable that the area of the shield 101 is minimised as far as possible, whilst still maintaining an acceptable level of coverage of the sweat gland area, in order that excess material does not reduce the comfort of the shield to the wearer.

Referring to the Figure, said perspiration shield comprises tab portion 105 extending outwardly of the natural periphery of the shield 101. As will be described, the primary function of tab portion 105 is to serve as a ‘handle’ to be gripped by the wearer to conveniently allow a user to break the adhesive bond between the shield and the skin and thus remove the shield 101 from the underarm skin.

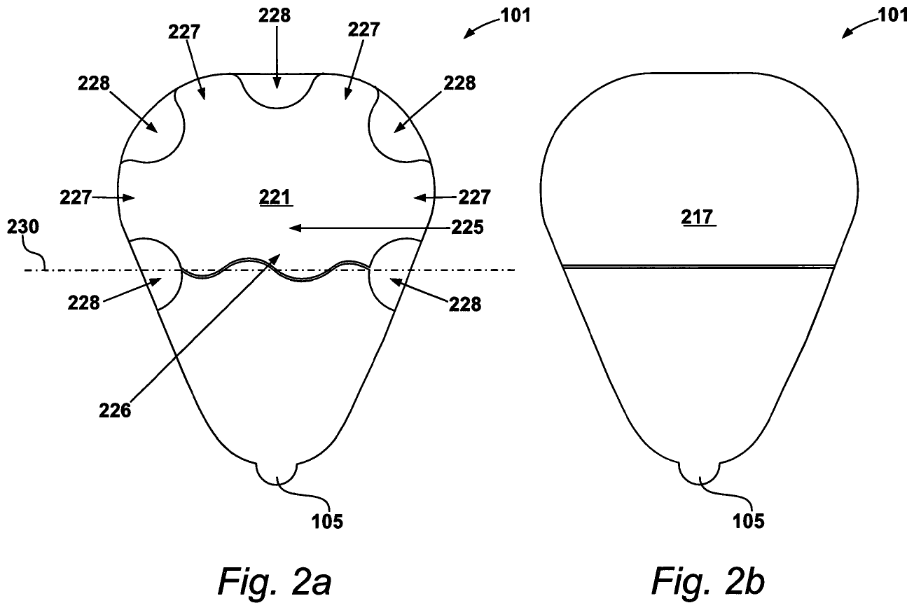

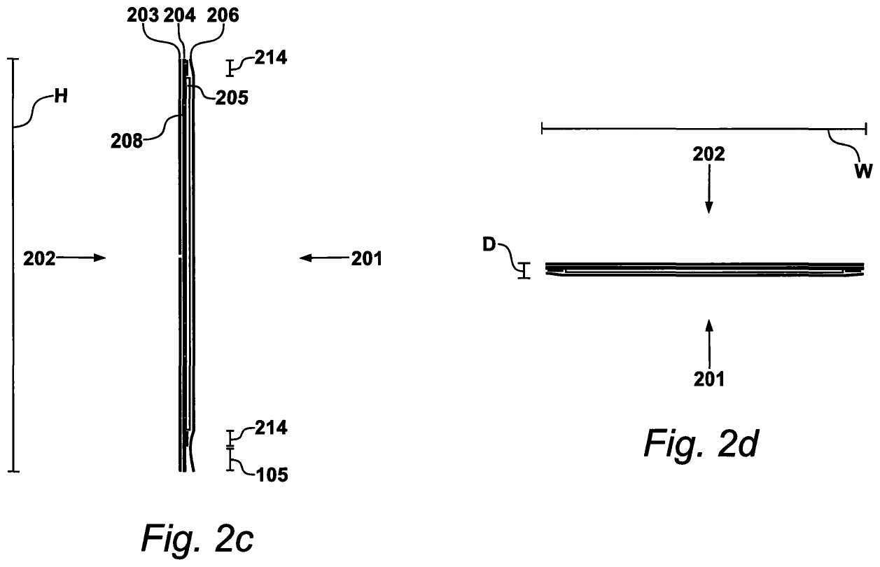

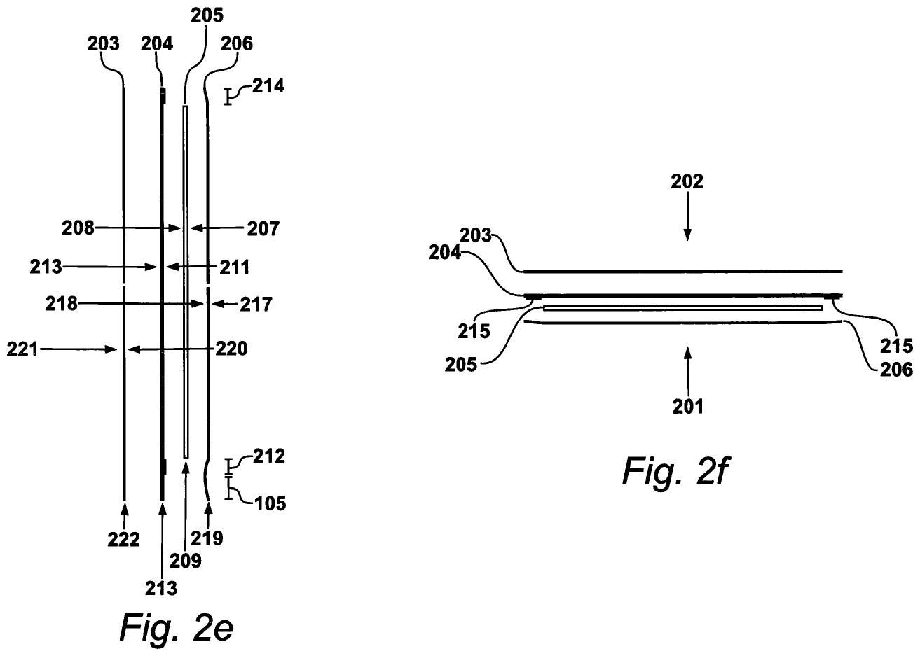

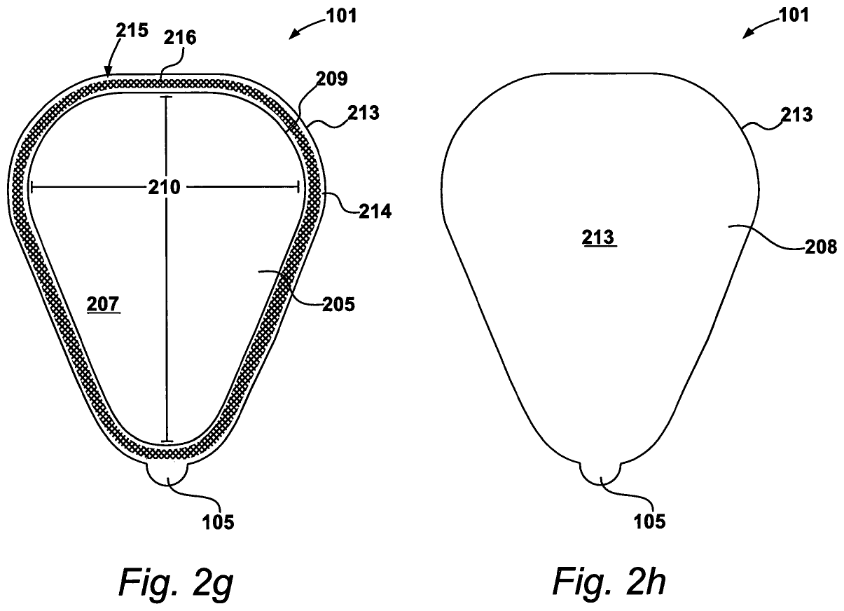

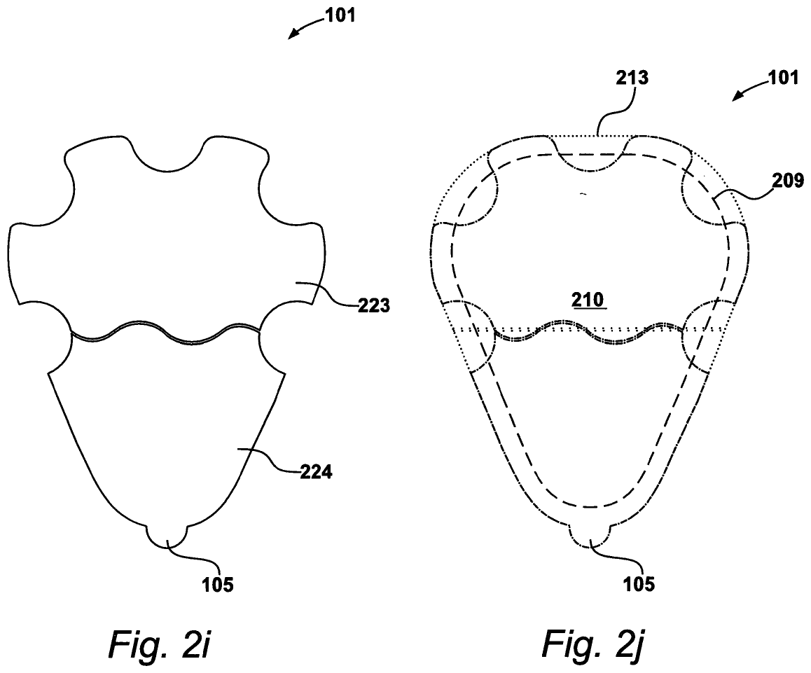

### FIGS. 2a, 2b, 2c, 2d, 2e, 2f, 2g, 2h, 2i & 2j

Perspiration shield 101 and its components are shown in isolation in FIGS. 2a, 2b, 2c, 2d, 2e, 2f, 2g, 2h, 2i & 2j. The shield is shown in outer and inner plan views in FIGS. 2a and 2b respectively, in face and end elevation views in FIGS. 2c and 2d respectively, in face and end elevation exploded views in FIGS. 2e and 2f respectively, and in partial inner and outer plan views in FIGS. 2g and 2h respectively. The reinforcing layer of the perspiration shield is shown in a plan view in FIG. 2i, and the layers of the perspiration shield are illustrated schematically in a plan view in FIG. 2j.

In the embodiment said perspiration shield 101 is substantially planar defining an inner face 201 configured to face towards and contact the skin of the underarm when in use, and an opposing outer face 202 configured to face away from the skin when the shield is in use. Said shield 101 comprises principally of a layer of perspiration absorbent material for absorbing perspiration, a perspiration impermeable layer for containing the absorbed perspiration and resultant odorous gases, a reinforcing layer which reinforces the perspiration impermeable layer during application of the shield to the skin to prevent excessive flexing, wrinkling or creasing of the perspiration impermeable layer during application, an adhesive composition disposed on the perspiration impermeable layer for releasably adhering the shield to the skin of the underarm, and a liner layer for covering the adhesive composition and perspiration absorbent material prior to application of the shield to thereby prevent contamination of the adhesive composition or perspiration absorbent material prior to application of the shield to the skin.

Referring to the figures, in the specific embodiment said shield comprises four depth wise spaced layers in the form of reinforcing layer 203, perspiration impermeable layer 204, perspiration absorbent layer 205, and liner layer 206. As will be described, said liner layer 206 and said reinforcing layer 203 are intended for separation from said perspiration impermeable layer 204 and said perspiration absorbent layer 205 prior to and following respectively, adhering of the perspiration impermeable layer to the skin of a wearer's underarm.

Referring in particular to FIGS. 2c and 2d, in the embodiment said perspiration absorbent material defines a perspiration absorbent layer 205 having an inner face 207 and an outer face 208, and a peripheral edge 209. In the embodiment, said perspiration absorbent layer 205 is generally acute-isosceles triangular in shape and defines a first area 210 of the shield 101, which first area is exposed in use on the inner face 201 of the shield 101. Exposing the perspiration absorbent material on the inner face 201 of the shield 101, i.e. not covering the perspiration absorbent material with a cover material such as an adhesive composition, advantageously improves its absorption of perspiration. In the embodiment, said perspiration absorbent layer 205 comprises a blend of a semi-synthetic viscose fibre (viscose rayon), and a type of synthetic fibre referred to by the trade mark Super Absorbent Fibre, formed into a ‘pad’ with a thickness of approximately 50 micro metres.

In the embodiment said perspiration impermeable layer 204 is distinct from said perspiration absorbent layer 205, it having an inner face 211 and an outer face 212, and having a peripheral edge 213 defining a second shape. In the embodiment, said perspiration impermeable layer 204 is also generally acute isosceles triangular in shape, and has a relatively larger area than the area of said perspiration absorbent layer 205. In the embodiment, said perspiration impermeable layer 204 comprises a polyurethane (PU) polymer material. More particularly, in the specific embodiment, said perspiration impermeable layer 204 comprises a flexible sheet of PU material with a substantially uniform thickness of approximately 30 micro metres.

Referring to the Figures, in the embodiment said perspiration absorbent layer 205 comprising a perspiration absorbent material is arranged on the inner face 211 of said perspiration impermeable layer 204, such that the outer face 206 of said perspiration absorbent layer 205 is in contact with the inner face 211 of said perspiration impermeable layer 204. Preferably the perspiration absorbent layer 205 is permanently attached to the inner face 211 of the perspiration impermeable layer 204 in a way that said perspiration absorbent layer 205 is substantially prevented from detaching therefrom during normal use of the shield 101. In the specific embodiment, the perspiration absorbent layer 205 is bonded to the perspiration impermeable layer 204 by way of a setting adhesive composition applied over substantially the full area of the outer face 208 of the perspiration absorbent layer 205.

As previously described, in the specific embodiment said perspiration absorbent layer 205 and said perspiration impermeable layer 204 are each generally acute-isosceles triangular in shape, said perspiration impermeable layer 204 however having a relatively greater area than said perspiration absorbent layer 205. Moreover, in the embodiment said perspiration absorbent layer 205 and said perspiration impermeable layer 204 are relatively oriented such that the bases of each generally acute isosceles triangular layer are substantially mutually aligned, as are the vertexes of each layer. Thus, as illustrated, when said perspiration absorbent layer 205 is so arranged with respect to said perspiration impermeable layer 204, a border region 214 is defined between the peripheral edge 209 of said perspiration absorbent layer 205 and the peripheral edge 213 of said perspiration impermeable layer 204. The border region 214 is defined by the inner face 211 of said perspiration impermeable layer 204 extending adjacent substantially the full circumference of the perspiration impermeable layer 204. In the embodiment, the border region 214 is substantially uniform in width about the circumference of the perspiration impermeable layer 204. That is to say, the radial distance between the peripheral edge 209 of the perspiration absorbent layer 205 and the peripheral edge 213 of the perspiration impermeable layer 204 is substantially uniform about the circumference of the shield.

An adhesive composition 215 is disposed on the inner face of said shield 101 about said border region 214 defining an adhesive border 216. The adhesive composition 215 is configured to releasably adhere the shield 101 to the skin of a wearer's underarm. More particularly, the adhesive composition 215 is disposed on the inner face 213 of said perspiration impermeable layer 204 proximal the peripheral edge 213 of the perspiration impermeable layer 204. In the embodiment, the adhesive composition 215 is disposed over substantially the full area of the border region 214. Thus, in the embodiment, said adhesive border 216 is substantially uniform in width about the circumference of the shield, occupying substantially the full distance between the peripheral edge 209 of the perspiration absorbent layer 205 and the peripheral edge 213 of the perspiration impermeable layer 204.

Referring to the Figures and in particular FIG. 2a, in the embodiment said adhesive border 216 defines a border extending substantially completely about the circumference of the first area 210 defined by the perspiration absorbent layer 205, the adhesive border 216 defining a generally triangular annulus extending about the first area 210 without overlapping the first area 210. Moreover, in the embodiment, said adhesive border 216 extends circumferentially about the first area 210 substantially unbroken. That is to say, the adhesive border 216 is substantially continuous about the circumference of the shield 101. One advantage of the adhesive border being unbroken is that perspiration absorbed by the perspiration absorbent layer 205 of perspiration absorbent material is contained between the shield 101 and the wearer's skin in the perspiration absorbent layer 205, and the perspiration is prevented from leaching outwardly of the shield as might occur if the adhesive border 216 were broken. Moreover, the continuous border of adhesive serves to prevent release of odorant substances resulting from bacterial activity in the perspiration.

It is required that the adhesive composition adheres securely to the skin of a user's underarm so as to prevent accidental detachment of the shield during use, or otherwise leakage of perspiration past the shield. However, it is desirable that the adhesive composition does not irritate the wearer's underarm skin during the relatively prolonged periods during which shield 101 is anticipated to be adhered to the underarm. Perspiration shields for adhering to the skin are known which use an acrylic adhesive, which type of adhesive has often been preferred for such applications due to its low cost, good adherence to skin, and chemical stability. However, it has been found that acrylic adhesives can tend to cause irritation and sensitisation to the skin. Accordingly, in the specific embodiment adhesive composition 215 comprises principally of a silicone based adhesive substance. It has been found that a silicone based adhesive adheres sufficiently to the wearer's skin, whilst avoiding irritation of the skin.

As previously described, in the embodiment, said shield 101 further comprises a liner layer 206 having an inner face 217, an outer face 218, and a peripheral edge 219. Said liner layer is arranged innermost of said shield such that the outer face 218 thereof is in contact with said inner face 211 of said perspiration impermeable layer 204 and said inner face of said perspiration absorbent layer 205. In the illustrated embodiment, the liner layer is configured to extend substantially across the full area of the perspiration impermeable layer 204, covering both the perspiration absorbent material 205 arranged thereon, and the adhesive region 214. The liner layer 206 covers the inner face 207 of the perspiration absorbent layer 205, thus preventing contamination of the inner face 207 thereof prior to use of the shield. Secondly, the liner layer 206 covers the adhesive region 214 of the perspiration impermeable layer 204, thereby preventing contamination of the adhesive substance prior to use, and so preserving its adhesive properties.

In the embodiment, the liner layer 206 is formed in two sections from a flexible sheet of an ethylene based plastics material with a thickness in the range of 5 micro metres to 50 micro metres. In the specific embodiment, the liner layer sections each comprise a flexible sheet of low-density polyethylene (LDPE) with a thickness of approximately 20 micro metre. The outer face 218 of said liner layer 206 is substantially free of any coating or contamination, thus the outer face 218 thereof is defined by the exposed LDPE sheet. Ethylene based plastics, and in particularly LDPE plastic, is a preferred material for the construction of said liner layer 206 for the primary reasons that it may be readily separated from the adhesive composition 215 disposed on the perspiration impermeable layer 204. Further, it has been found that silicone based adhesive compositions, such as adhesive composition 215 applied to the inner face 211 of said perspiration impermeable layer 204, are not excessively absorbed by the LDPE material. Contrarily, it has been found that conventional materials from which release liners are formed, for example, clay coated paper, can have a tendency to absorb silicone based adhesives if left in contact during prolonged storage periods.

Referring still to the Figures, in the specific embodiment said perspiration shield 101 further comprises a reinforcing layer 203, the reinforcing layer 203 having an inner face 220, an outer face 221, and a peripheral edge 222. As shown in the Figures, reinforcing layer 203 is arranged to extend across said outer face 212 of said perspiration impermeable layer 204, with said inner face 220 of said reinforcing layer 203 in contact with said outer face 212 of said perspiration impermeable layer 204. In the specific embodiment, said reinforcing layer is formed of two flexible layer sections, 223 and 224, each formed of a sheet of clay coated paper having a thickness of approximately 30 micro meters.

As previously described, the principle purpose of said reinforcing layer is to stiffen the relatively flexible PU perspiration impermeable layer 204 prior to application of the shield, to thereby prevent excessive flexing, wrinkling or creasing of the perspiration impermeable layer during application.

As previously described, it is desirable that said perspiration impermeable layer is relatively thin and flexible, primarily so as to improve comfort of the shield 101 when being worn, and also to allow the shield to maintain close conformation to the skin of the wearer's underarm even as the wearer moves. However it will be appreciated that, although advantageous in use, the preferred high degree of flexibility of said perspiration impermeable layer 204 can be problematic when applying the shield to the skin, as the perspiration impermeable layer may tend to crease or wrinkle, and so adhere to itself, or otherwise adhere to a wrong location of the wearer's underarm. Thus, said reinforcing layer 203 is releasably attached to said perspiration impermeable layer 204 to thereby stiffen the perspiration impermeable layer to improve application of the shield to the skin.

Referring to the figures, in the embodiment said reinforcing layer 203 has an irregular shape, rather than being shaped to match said perspiration impermeable layer 204. More particularly, in the embodiment said reinforcing layer has a generally ‘star’ shape, defining a main portion, indicated generally at 225, centred generally on the centre point 226 of the reinforcing layer 203, from which a plurality of radially extending regions 227 extend. Radially concaved regions 228 are defined between the radially extending regions 227. As illustrated, the radially extending regions 227 extend outwardly of the centre 226 of the reinforcing layer 203 to a greater extent than said radially concaved regions 228. Specifically, in the embodiment, said reinforcing layer 203 is shaped and sized such that radially extending regions 227 extend outwardly of the centre 221 of the reinforcing layer to an extent such that the peripheral edge 222 thereof at the greatest extent of the radially protruding regions is substantially depth wise aligned with the peripheral edge 213 of said perspiration impermeable layer 204. Moreover, in between said radially protruding regions 227, said radially concaved regions 228 are defined by the peripheral edge 222 which extends outwardly of the centre 226 of the reinforcing layer 203 to a minimum extent that said peripheral edge 222 is depth wise aligned with a portion of said perspiration impermeable layer 204 that is located radially inward of said peripheral edge 213 thereof.

Preferably, said reinforcing layer 203 is shaped such that the peripheral edge 222 of said reinforcing layer adjacent the maximum extent of said radially protruding regions 227 extends radially further outwardly of the centre 226 than the peripheral edge 222 adjacent the minimum extent of said radially concaved regions 228 by a radial distance in the range of 2 millimetre to 40 millimetre, and more preferably in the range of 5 to 20 millimetre, most preferable approximately 10 millimetre.

As will be appreciated, during application of said shield 101 to the wearer's underarm skin, it is desirable that the perspiration impermeable layer and the perspiration absorbent layer are allowed to flex to an extent to allow close conformation of the shield to the underarm skin, and most preferably to allow close contact between the whole area of the inner surface of the perspiration absorbent layer 205 and the wearer's underarm skin. Thus, it is desirable that, whilst being prevented from excessively creasing or wrinkling, the perspiration impermeable layer 204 and the perspiration absorbent layer 205 are allowed to controllably flex.

As will be appreciated, said radially protruding regions 227 extend substantially the full distance to the peripheral edge 213 of the perspiration impermeable layer 204, thus allowing releasable attachment of the reinforcing layer 203 to the perspiration impermeable layer 204 adjacent the peripheral edge 213 thereof, thereby supporting the perspiration impermeable layer across its full width and height and preventing its creasing/wrinkling. As illustrated though, said radially concaved regions 228 do not extend fully to be depth wise aligned with the peripheral edge 213 of the perspiration impermeable layer 204. Rather, as illustrated, the peripheral edge 219 of said reinforcing layer 211 adjacent each said radially concaved region 228 is aligned with a position on the perspiration impermeable layer 204 radially inward of the peripheral edge 211 thereof.

As will be understood, said radially concaved regions 228 improve the flexibility of the reinforcing layer 203, and so the perspiration impermeable layer 204, thus allowing the shield to controllably flex during application to conform to the shape of the wearer's underarm. In the specific embodiment, as illustrated, a pair of said radially concaved regions 228 are height wise aligned along an axis 224 extending width wise of the shield 101 approximately halfway the height of the shield along opposed height wise extending sides of the perspiration impermeable layer 204. The aligned radially concaved region pair 228 improve the flexibility of the reinforcing layer 203, and so the perspiration impermeable layer 204, about the axis 230.

In the embodiment, said reinforcing layer 203 is attached to said perspiration impermeable layer 204 across substantially the whole area of said inner face 220 of said reinforcing layer 203. In the specific embodiment, said reinforcing layer 203 is releasably attached to said perspiration impermeable layer 204 in a way as to allow said reinforcing layer to be readily separated from said perspiration impermeable layer 204 following application to the skin. In the embodiment the reinforcing layer 203 is attached to the perspiration impermeable layer 204 by way of a pressure sensitive adhesive applied across substantially the whole area of said inner face 220 of said reinforcing layer. Said pressure sensitive adhesive is selected so as to provide the required degree of adhesion between the reinforcing layer 203 and the perspiration impermeable layer 204, whilst allowing for ready separation of said reinforcing layer 203 from said perspiration impermeable layer 204 without causing damage to said perspiration impermeable layer 204, or otherwise leaving adhesive residue on said outer face 212 of said perspiration impermeable layer 204.

Through the above noted configuration of said reinforcing layer 203, said reinforcing layer is able to impart the required degree of stiffness to said perspiration impermeable layer such that wrinkling or creasing of said perspiration impermeable layer 204 is substantially prevented or at least inhibited, without preventing said perspiration impermeable layer 204, or indeed said shield 101 as a whole, from desirably flexing.

Referring particularly to FIGS. 2a and 2b, as previously described, in the embodiment said shield 101 comprises a tab portion 105 configured to facilitate removal of the shield 101 from the skin of the underarm of the wearer. Said tab portion 101 extends radially outwardly of said border region 214 on which said adhesive border 214 is disposed. In the specific embodiment, said tab portion 105 is defined by said perspiration impermeable layer 204. Thus, in the embodiment, said tab portion 105 is defined by the peripheral edge 213 of said perspiration impermeable layer 204 at a region where the radial distance between the peripheral edge 209 of said perspiration absorbent layer 205 and the peripheral edge 213 of said perspiration impermeable layer 204 is increased to greater than the average distance between the peripheral edge 209 and the peripheral edge 213.

As illustrated, said tab portion 105 extends outwardly of the natural periphery of the main part of the shield defined by the peripheral edge 213 of the perspiration impermeable layer 204. In the embodiment said tab portion 105 extends radially outwardly of the adhesive border 216 adjacent the aligned vertexes of the perspiration absorbent layer 205 and the perspiration impermeable layer 204. That is to say, the tab portion 105 extends outwardly adjacent the torso end 104 of the shield.

In the embodiment, said tab portion 105 is substantially free of said adhesive composition 215. More particularly, in the embodiment the inner (skin-facing) face of said tab portion 105 is free of any adhesive composition. An advantage of the inner face of said tab portion being substantially free of any adhesive is that the tab portion 105 itself will not be adhered to the wearer's skin when the shield is adhered to the skin as described. That the tab portion 105 is substantially free of adhesive on its inner face and so will not adhere to the skin has the advantage that the tab portion 105 can be easily gripped by a wearer to allow removal of the shield from the underarm skin.

In the embodiment the tab portion 105 is shaped and dimensioned so as to be sufficiently large as to allow a wearer to grip the tab portion, but not so large as to substantially reduce the comfort of the shield 101. In the specific embodiment, the tab portion 105 is dimensioned as to conveniently allow a wearer to obtain sufficient purchase on the tab portion between the thumb and forefinger as to exert a force on the tab portion sufficient to break the adhesive bond between the shield 101 and the skin of the wearer's underarm.

In preferred embodiments, said tab portion 105 is significantly smaller than the main part of said shield 101. More particularly, preferably the area of said tab portion 105 is significantly less than said first area 210 defined by said perspiration absorbent layer 205 of perspiration absorbent material.

It is preferable however that the tab portion 105 extends outwardly of the adhesive border 216 to a distance that is substantially equal to or greater than the width of said adhesive border 216. That is to say, it is preferable that the tab portion 105 extends outwardly of the adhesive border by a radial distance that is substantially equal to or greater than the radial distance between said peripheral edge 209 and said peripheral edge 213. This relative dimension is advantageous as it would be expected to allow the wearer to obtain sufficient purchase on the tab portion as to overcome the adhesive bond between shield 101 and underarm skin.

In the illustrated embodiment, the height ‘H’ of the shield 101 is approximately 105 millimetre, the width ‘W’ is approximately 80 millimetre, and the depth ‘D’ is approximately 2 millimetre.

It will be understood by the skilled person that a number of different materials are suitable for constructing the shield 101. For example, in alternative embodiments, said perspiration absorbing layer may be formed of alternative materials capable of absorbing sufficient volumes of perspiration, for example, cotton fibre or paper pulp materials. The primary requirement of said perspiration absorbent material is that it is capable of absorbing volumes of perspiration incident thereon as it might be expected the wearer will excrete from the underarm in a single day. As an example, the preferred perspiration absorbent material has a perspiration absorbency of approximately 30 grams of perspiration per gram of dry absorbent material. It will further be appreciated that alternative embodiments of the invention may utilise alternative perspiration impermeable materials, for example, other plastic films or metallic foils. Appreciating that no material can ever be considered entirely resistant to the passage of liquid, the perspiration impermeable material selected should ideally substantially prevent the passage of perspiration there-through in the volumes and conditions as might be expected to be experienced when the shield is adhered to the wearer's underarm.

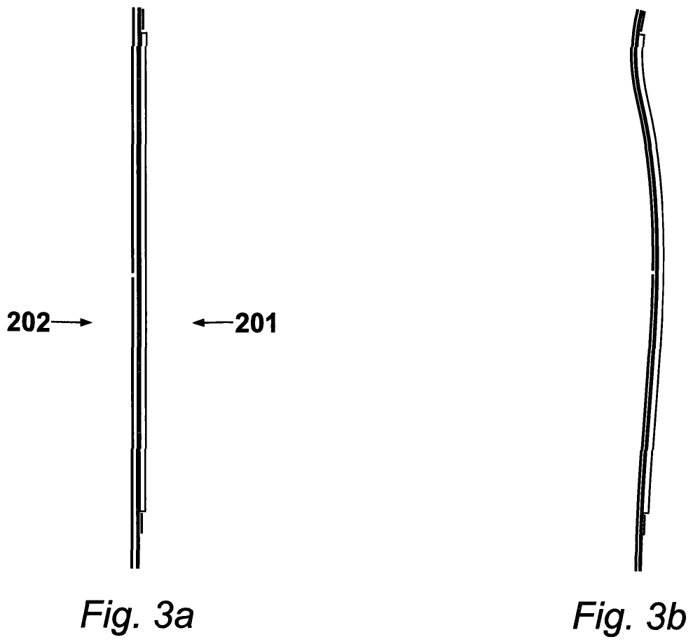

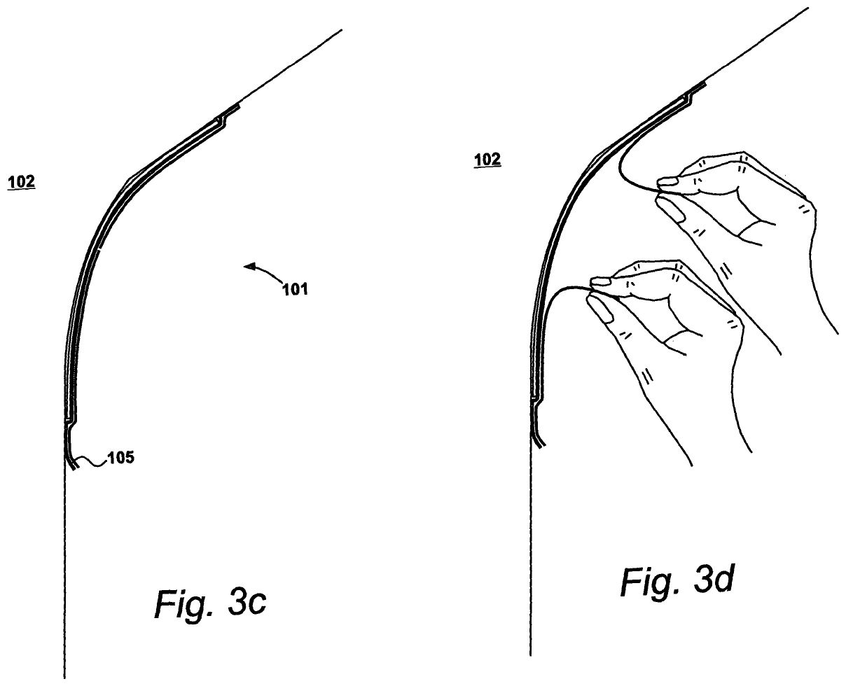

### FIGS. 3a, 3b, 3c & 3d.

Perspiration shield 101 is shown in a first and second side elevation views in FIGS. 3a and 3b, is shown attached to a wearer's underarm in FIG. 3c with the reinforcing layer attached, and in FIG. 3d is shown attached to the wearer's underarm with the reinforcing layer partially detached.

Referring to the Figures, in the illustrated condition said liner layer 206 has been removed from contact with the inner face 211 of the perspiration impermeable layer 204 and the inner face 207 of the perspiration absorbent layer 205. Thus, the shield 101 is in a condition ready for attachment to the skin of a wearer's underarm.

As previously described, in the embodiment, the perspiration shield 101 is configured to be relatively flexible. Thus, in the embodiment, said perspiration absorbent layer 205, said perspiration impermeable layer 204, and said reinforcing layer 203 are selected as to be relatively flexible rather than rigid.

In the specific embodiment, the perspiration shield 101 is resiliently biased towards an un-flexed condition in which the perspiration shield is substantially planar (as shown in FIGS. 2a to 2j). The resilient biasing of the perspiration shield towards the planar condition advantageously resists creasing or wrinkling of the shield in use.

However, as noted, in the embodiment the perspiration shield 101 is resiliently flexible away from the planar condition, in order to allow the shield to be flexed to match the contours of a wearer's underarm. Generally, the shield 101 is resiliently flexible at least about a height wise extending axis, which extends in the height direction H, and about a width wise extending axis, which extends in the width W direction.

However, whilst the various layers of the shield are selected and arranged such that the shield may be relatively flexible, as previously described, it is desirable that the perspiration impermeable layer is prevented from excessively creasing or wrinkling during application. Thus, as shown in the Figures, said reinforcing layer 203 is adapted to allow the perspiration impermeable layer 204 and the perspiration absorbent layer 205 to flex to an extent, but to also prevent wrinkling/creasing of the perspiration impermeable layer, and in particular to prevent creasing or wrinkling of the region of the perspiration impermeable layer to which the adhesive composition 215 is applied.

Thus, the shield 101 has an inherent degree of flexibility allowing the shield to be readily shaped to conform closely to the shape of a wearer's underarm. Moreover, the flexibility of the materials used in the construction of the shield all it to flex in response to movement by the wearer. The flexibility of the shield 101 thus improves comfort to the wearer.

Referring to FIGS. 3c and 3d, perspiration shield 101 is shown fully adhered to the underarm 401 of the wearer 102. Thus, as illustrated, in the fully adhered condition the adhesive border 216 adheres the peripheral edge of the perspiration impermeable layer 204 to the skin. Said perspiration absorbent layer 205 is thus maintained in close contact with the sweat gland area of the wearer's underarm, with said adhesive border 216 extending generally about the periphery of the sweat gland area. As illustrated, said tab portion 105 remains un-adhered to the skin.

As previously described, said reinforcing layer 203 is intended to remain in place attached to the outer surface 213 of the perspiration impermeable layer 204 during application of the shield to the wearer's underarm, as shown in FIG. 3c. The reinforcing layer 203 is then intended to be detached from the perspiration impermeable layer 204 once the shield has been adhered to the wearer's underarm skin, by peeling the two sections of the reinforcing layer from the outer face 213 of the perspiration impermeable layer, as shown in FIG. 3d.

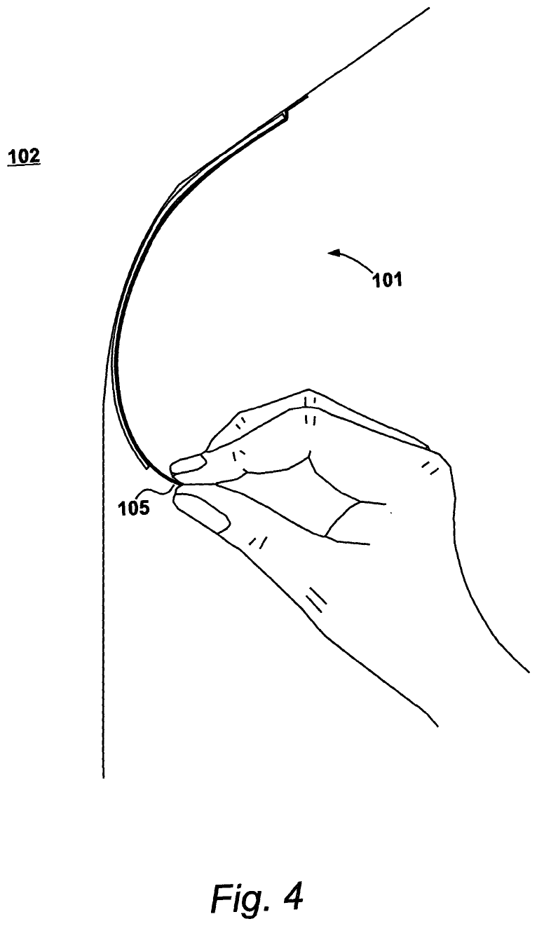

### FIG. 4

Perspiration shield 101 is shown adhered to the underarm skin of the wearer in a cross-sectional view in FIG. 4.

Referring to FIG. 4, the shield 101 is shown partially removed from the skin. As illustrated, tab portion 105 is configured such that the wearer may grip the tab portion 105 between thumb and forefinger to thereby remove the shield from the skin of the underarm.

In the specific embodiment, the shield 101 is configured to be single-use. Thus, in the embodiment it is expected that the shield 101 will be removed from the skin and disposed of following use.

1. A perspiration shield releasably adherable to the skin of an underarm, the shield having an inner face and an outer face, the shield comprising:
a perspiration impermeable layer having an inner face, an outer face, and a peripheral edge, and comprising a silicone based adhesive suitable for releasably adhering said shield to the skin of an underarm, said adhesive being disposed on said inner face of the perspiration impermeable layer adjacent its peripheral edge and forming an adhesive border;
a perspiration absorbent material arranged on the inner face of said perspiration impermeable layer and within the adhesive border; and
a liner layer having an inner face, an outer face, and a peripheral edge, said liner layer being arranged such that its outer face is in contact with said inner face of said perspiration impermeable layer and extends substantially fully across said adhesive border thereof so as to substantially cover said adhesive border thereof, said liner layer comprises low-density polyethylene (LDPE) material.

2. The perspiration shield as claimed in claim 1, in which radially protruding regions of a reinforcing layer extend radially outwardly of a centre of the reinforcing layer to an extent that a peripheral edge of said reinforcing layer at the greatest extent of said radially protruding regions is substantially aligned with said peripheral edge of said perspiration impermeable layer.

3. The perspiration shield as claimed in claim 1, in which radially concaved regions of a reinforcing layer extend radially outwardly of a centre of the reinforcing layer to an extent that a peripheral edge of said reinforcing layer at the minimum extent of said radially concaved regions is aligned with a part of said perspiration impermeable layer radially inward of said peripheral edge of said perspiration impermeable layer.

4. (canceled)

5. The perspiration shield as claimed in claim 1, in which an inner face of a reinforcing layer is in contact with and releasably attached to said outer face of said perspiration impermeable layer adjacent a peripheral edge of said reinforcing layer.

6. The perspiration shield as claimed in claim 1, in which an inner face of a reinforcing layer is in contact with and releasably attached to said outer face of said perspiration impermeable layer adjacent a peripheral edge of said reinforcing layer at the maximum extent of radially protruding regions of said reinforcing layer adjacent the peripheral edge of said perspiration impermeable layer.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The perspiration shield of claim 1, in which said perspiration impermeable layer comprises a polyurethane film.

22. The perspiration shield of claim 1, in which said perspiration absorbent material comprises viscose fibre.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A perspiration shield releasably adherable to the skin of an underarm, the shield having an inner face and an outer face, the shield comprising:
a perspiration impermeable layer having an inner face, an outer face, and a peripheral edge, and comprising a silicone based adhesive suitable for releasably adhering said shield to the skin of an underarm, said adhesive being disposed on said inner face of the perspiration impermeable layer adjacent its peripheral edge and forming an adhesive border;
a perspiration absorbent material arranged on the inner face of said perspiration impermeable layer and within the adhesive border;
a liner layer having an inner face, an outer face, and a peripheral edge, said liner layer being arranged such that its outer face is in contact with said inner face of said perspiration impermeable layer and extends substantially fully across said adhesive border thereof so as to substantially cover said adhesive border thereof, said liner layer comprises low-density polyethylene (LDPE) material; and
a reinforcing layer having an inner face, an outer face, and a peripheral edge, said reinforcing layer being arranged on said outer face of said perspiration impermeable layer and extending at least partly thereacross, with at least a portion of said inner face of said reinforcing layer in contact with and releasably attached to said outer face of said perspiration impermeable layer; in which
said reinforcing layer defines radially protruding regions and radially concaved regions bounded by said peripheral edge thereof, said radially protruding regions extending radially outwardly of a centre of said reinforcing layer to a greater extent than said radially concaved regions.

40. The perspiration shield as claimed in claim 39, in which said liner layer and said perspiration impermeable layer are adapted so as to be readily separable.

41. The perspiration shield as claimed in claim 39, in which said reinforcing layer is relatively stiffer than said perspiration impermeable layer.

42. The perspiration shield as claimed in claim 39, in which said perspiration impermeable layer is approximately a polygon in shape, the peripheral edge thereof defining generally faces and vertices forming a closed loop.

43. The perspiration shield as claimed in claim 39, in which said reinforcing layer and said perspiration impermeable layer are relatively oriented such that at least one of said radially concaved regions of said reinforcing layer is aligned with at least one of said vertices of said perspiration impermeable layer.

44. The perspiration shield as claimed in claim 39, in which said reinforcing layer comprises first and second discrete portions.

45. The perspiration shield as claimed in claim 39, in which said perspiration absorbent material is permanently attached to said inner face of said perspiration impermeable material.

46. The perspiration shield as claimed in claim 39, in which said reinforcing layer is releasably attached to said perspiration impermeable layer by attachment means.

47. The perspiration shield as claimed in claim 39, in which said perspiration absorbent material defines a discrete layer of said shield.

48. The perspiration shield as claimed in claim 39, in which each of said perspiration impermeable layer, said perspiration absorbent layer, and said liner layer are generally planar flexible sheets.
